(19) United States
(12) Patent Application Publication
(10) Pub. No.: US 2025/0057761 A1
(43) Pub. Date: Feb. 20, 2025

(54) GASTRIC RESIDENCE ARTICLE FOR ORAL ADMINISTRATION

(71) Applicant: JUNION LABS PTE. LTD., Singapore (SG)

(72) Inventors: Jingnan LUO, Singapore (SG); Bo Huai Moses LEE, Singapore (SG)

(21) Appl. No.: 18/938,355

(22) Filed: Nov. 6, 2024

Related U.S. Application Data
(63) Continuation of application No. PCT/SG2023/050394, filed on Jun. 1, 2023.
(60) Provisional application No. 63/365,785, filed on Jun. 3, 2022.

(51) Int. Cl. A61K 9/00

(52) U.S. Cl. CPC A61K 9/0065

(57) ABSTRACT

A gastric residence article for oral administration is provided. The article includes a foldable central component, multiple elongated limb components and multiple linker components. The foldable central component includes multiple ribs that include at least one superelastic alloy. The elongated limb components are radially attached to the foldable central component, where the multiple elongated limb components correspond to the multiple ribs. Each of the linker components connects one of the elongated limb components to the foldable central component. The ribs are releasable from a folded first state to an unfolded second state. At least one of the elongated limb components is configured to releasably store drugs. The linker components are configured to transform from a rigid first state to a disengaged second state to allow the elongated limb component to move independently with respect to the foldable central component.

### CROSS REFERENCE TO RELATED APPLICATION

This application claims priority of the International Patent Application No. PCT/SG2023/050394, filed on Jun. 1, 2023, which claims the priority of U.S. Patent Application No. 63/365,785 entitled “A GASTRIC RESIDENCE ARTICLE FOR ORAL ADMINISTRATION” filed with the United States Patent And Trademark Office on Jun. 3, 2022, which is incorporated herein by reference in its entirety.

### TECHNICAL FIELD

The present invention relates to a gastric residence article for oral administration.

### BACKGROUND

Medical adherence to drug regimens over an extended duration is often poor. Low adherence rates are greatest in primary and secondary prevention when a disease to be prevented or treated is often asymptomatic and the drug regimens have no immediate tangible effects. Current approaches to improving adherence rates such as educational interventions and counseling have achieved only modest improvements.

Pharmacologic solutions such as invasive delivery methods and pharmacologic agents are often less well received due their invasive nature, while oral administration of a pharmaceutical drug is more widely accepted as it is easier and less expensive. However, the transit time through the human gastrointestinal tract is only about 24 to 48 hours, including about 1 to 2 hours in the stomach, about 3 hours in the small intestine, and about 6 to 12 hours in the large intestine. Therefore, a single administration of the drug usually cannot achieve a dosage frequency prescribed over a duration longer than the transit time.

One approach for oral-administered dosage forms to achieve the dosage frequency over an extended duration is by including a self-expanding mechanism into the dosage form which physically prevents the dosage form from leaving the gastric chamber. Conventionally, the self-expanding mechanism is made entirely of a super-elastic material such as elastomers. However, there are several drawbacks associated with using only polymers to produce the mechanism.

It should be noted that for the dosage form to prevent itself from leaving the gastric chamber, it needs to have a minimum flexural modulus to resist being pushed through the pyloric sphincter that connect the stomach to the duodenum. Polymers have relatively low flexural modulus and thus, if polymers were used in the self-expanding mechanism, there is a limit as to how small the mechanism can be because the cross section of the mechanism would need to be large enough to achieve the minimum flexural modulus. This limits the minimum size of the drug which causes the drug to be unsuitable for patients with swallowing difficulties or young children.

Another drawback of using polymers as the shape memory material is the limited pool of polymers that meet the requirement for minimum flexural modulus, while also meeting other material properties requirements such as being biocompatible, being melt processable to allow for low-cost mass production, having sufficient surface energy to bond with other parts of the dosage form and having a high creep resistance for the self-expanding mechanism to spring back to its initial shape after long term storage.

A need therefore exists to address at least one of the problems above or to provide a useful alternative.

### SUMMARY

According to a first aspect of the present invention, there is provided a gastric residence article for oral administration comprising:
a foldable central superelastic alloy component;
a plurality of elongated limb components loadable with an active substance, the active substance being a therapeutic agent or a diagnostic agent,
wherein the plurality of elongated limb components are each connected to the foldable central superelastic alloy component via a linker component,
wherein the foldable central superelastic alloy component undergoes elastic deformation when the gastric residence article is in a folded configuration and recoils when the gastric residence article assumes an expanded configuration, and
wherein said linker component degrades, dissolves, disassociates, or mechanically weakens, thereby resulting in loss of a shape of the gastric residence article in the expanded configuration.

The expanded configuration of the gastric residence article may have a folding force of at least 0.5 N.

The expanded configuration of the gastric residence article may have an opening diameter of at least 2 cm.

The gastric residence article may be configured to retain the shape in the expanded configuration for at least 24 hours.

The foldable central superelastic alloy component may comprise at least one selected from a group consisting of Nitinol (Ni—Ti), Brass (Cu—Zn), copper-aluminium-nickel (Cu—Al—Ni) alloy, gold-cadmium (Au—Cd) alloy, gold-copper-zinc (Au—Cu—Zn) alloy, indium-thallium (In—Tl) alloy, cobalt-nickel-aluminium (Co—Ni—Al) alloy, cobalt-nickel-gallium (Co—Ni—Ga) alloy, copper-aluminium-beryllium-zirconium (Cu—Al—Be—Zr), copper-aluminium-beryllium-chromium (Cu—Al—Be—Cr) alloy, copper-aluminium-beryllium-gadolinium (Cu—Al—Be—Gd) alloy, copper-aluminium-nickel-hafnium (Cu—Al—Ni—Hf) alloy, copper-tin (Cu—Sn) alloy, copper-zinc-silicon (Cu—Zn—Si) alloy, copper-zinc-aluminium (Cu—Zn—Al) alloy, copper-zinc-silicon (Cu—Zn—Sn) alloy, iron-manganese-silicon (Fe—Mn—Si) alloy, iron-platinum (Fe—Pt) alloy, manganese-copper (Mn—Cu) alloy, nickel-iron-gallium (Ni—Fe—Ga) alloy, (Ni—Ti—Hf) alloy, nickel-titanium-palladium (Ni—Ti—Pd) alloy, nickel-manganese-gallium (Ni—Mn—Ga) alloy, nickel-manganese-gallium-copper (Ni—Mn—Ga—Cu) alloy, nickel-manganese-gallium-cobalt (Ni—Mn—Ga—Co) alloy, titanium-niobium (Ti—Nb) alloy.

The foldable central superelastic alloy component may be securely received within an outer portion.

At least 80% of the folding force may be attributed to the foldable central superelastic alloy component.

The linker component may be coupled to the outer portion.

The outer portion may be made of at least one selected from a group consisting of polypropylene, polystyrene, polyvinyl chloride, synthetic rubber, phenol formaldehyde resin (or Bakelite), neoprene, nylon, polyacrylonitrile, PVB, silicone, acrylonitrile butadiene styrene, high density polyethylene, polycarbonate, nylon, acrylic, polyethylene terephthalate, polybutylene terephthalate, acetal, polyimide, polyurethane, and epoxy.

The gastric residence article may further comprise a casing for housing the gastric residence article in the folded configuration, wherein the casing is configured to degrades, dissolves, disassociates, or mechanically weakens to allow the gastric residence article to recoil and assume the expanded configuration.

According to a second aspect of the present invention, there is provided a gastric residence article for oral administration comprising:
a foldable central shape memory alloy component;
a plurality of elongated limb components loadable with an active substance, the active substance being a therapeutic agent or a diagnostic agent,
wherein the plurality of elongated limb components are each connected to the foldable central shape memory alloy component via a linker component,
wherein the foldable central shape memory alloy component undergoes a phase transformation in its crystal structure from the martensite phase to the austenite phase, thereby transforming the article from a folded configuration to an expanded configuration, and
wherein said linker component degrades, dissolves, disassociates, or mechanically weakens, thereby resulting in loss of a shape of the gastric residence article in the expanded configuration.

The expanded configuration of the gastric residence article may have a folding force of at least about 0.5 N.

The expanded configuration of the gastric residence article may have an opening diameter of at least 2 cm.

The gastric residence article may be configured to retain the shape in the expanded configuration for at least 24 hours.

The foldable central shape memory alloy component may comprise at least one selected from a group consisting of Ni—Ti, Ni—Ti—Hf, Ni—Ti—Pd, Ni—Fe—Ga, Ni—Mn—Ga, Ni—Mn—Ga—Cu, Ni—Mn—Ga—Co, Ag—Cd, Co—Ni—Al, Co—Ni—Ga, Cu—Al—Be—X (X: Zr, B, Cr, Gd), Cu—Al—Ni, Cu—Al—Ni—Hf, Cu—Sn, Cu—Zn, Cu—Zn—X (X=Si, Al, Sn), Fe—Mn—Si, Fe—Pt, Mn—Cu, Ti—Nb.

The foldable central shape memory alloy component may be securely received within an outer portion.

At least 80% of the folding force may be attributed to the foldable central shape memory alloy component.

The linker component may be coupled to the outer portion.

The outer portion may be made of at least one selected from a group consisting of polypropylene, polystyrene, polyvinyl chloride, synthetic rubber, phenol formaldehyde resin (or Bakelite), neoprene, nylon, polyacrylonitrile, PVB, silicone, acrylonitrile butadiene styrene, high density polyethylene, polycarbonate, nylon, acrylic, polyethylene terephthalate, polybutylene terephthalate, acetal, polyimide, polyurethane, and epoxy.

According to a third aspect of the present invention, there is provided a gastric residence article for oral administration comprising:
a foldable central component comprising a plurality of ribs, wherein the ribs comprise at least one superelastic alloy;
a plurality of elongated limb components radially attached to the foldable central component, the plurality of elongated limb components corresponding to the plurality of ribs; and
a plurality of linker components, each of the linker components connecting one of the elongated limb components to the foldable central component.
wherein the ribs are releasable from a folded first state to an unfolded second state for deploying the elongated limb components,
wherein at least one of the elongated limb components is configured to releasably store drugs, and

wherein the linker components are configured to transform from a rigid first state to a disengaged second state to allow the elongated limb component to move independently with respect to the foldable central component.

The disengaged second state may comprise the linker components being in a flexible state to allow bending of the elongated limb components with respect to the foldable central component.

The disengaged second state may comprise the linker components breaking apart to disconnect the elongated limb components from the foldable central component.

The linker components may comprise a material that undergoes hydrolysis to transform the linker components from the rigid first state to the disengaged second state.

The superelastic alloy may comprise at least one selected from a group consisting of Nitinol (Ni—Ti), Brass (Cu—Zn), copper-aluminium-nickel (Cu—Al—Ni) alloy, gold-cadmium (Au—Cd) alloy, gold-copper-zinc (Au—Cu—Zn) alloy, indium-thallium (In—Tl) alloy, cobalt-nickel-aluminium (Co—Ni—Al) alloy, cobalt-nickel-gallium (Co—Ni—Ga) alloy, copper-aluminium-beryllium-zirconium (Cu—Al—Be—Zr), copper-aluminium-beryllium-chromium (Cu—Al—Be—Cr) alloy, copper-aluminium-beryllium-gadolinium (Cu—Al—Be—Gd) alloy, copper-aluminium-nickel-hafnium (Cu—Al—Ni—Hf) alloy, copper-tin (Cu—Sn) alloy, copper-zinc-silicon (Cu—Zn—Si) alloy, copper-zinc-aluminium (Cu—Zn—Al) alloy, copper-zinc-silicon (Cu—Zn—Sn) alloy, iron-manganese-silicon (Fe—Mn—Si) alloy, iron-platinum (Fe—Pt) alloy, manganese-copper (Mn—Cu) alloy, nickel-iron-gallium (Ni—Fe—Ga) alloy, (Ni—Ti—Hf) alloy, nickel-titanium-palladium (Ni—Ti—Pd) alloy, nickel-manganese-gallium (Ni—Mn—Ga) alloy, nickel-manganese-gallium-copper (Ni—Mn—Ga—Cu) alloy, nickel-manganese-gallium-cobalt (Ni—Mn—Ga—Co) alloy, titanium-niobium (Ti—Nb) alloy.

The superelastic alloy may be a shape memory alloy.

The shape memory alloy may comprise at least one selected from a group consisting of Ni—Ti, Ni—Ti—Hf, Ni—Ti—Pd, Ni—Fe—Ga, Ni—Mn—Ga, Ni—Mn—Ga—Cu, Ni—Mn—Ga—Co, Ag—Cd, Co—Ni—Al, Co—Ni—Ga, Cu—Al—Be—X (X: Zr, B, Cr, Gd), Cu—Al—Ni, Cu—Al—Ni—Hf, Cu—Sn, Cu—Zn, Cu—Zn—X (X=Si, Al, Sn), Fe—Mn—Si, Fe—Pt, Mn—Cu, Ti—Nb.

The foldable central component may further comprise an outer portion and wherein the ribs are securely received within the outer portion.

The linker components may connect each of the elongated limb components to the outer portion.

The outer portion may be made of at least one selected from a group consisting of polypropylene, polystyrene, polyvinyl chloride, synthetic rubber, phenol formaldehyde resin (or Bakelite), neoprene, nylon, polyacrylonitrile, PVB, silicone, acrylonitrile butadiene styrene, high density polyethylene, polycarbonate, nylon, acrylic, polyethylene terephthalate, polybutylene terephthalate, acetal, polyimide, polyurethane, and epoxy.

The gastric residence article may further comprise a casing for housing the foldable central component and the elongated limb components, wherein the casing is configured to be dissolved to release the ribs from the first folded state to the unfolded second state for deploying the elongated limb components.

The foldable central superelastic alloy component comprises a single piece.

The single piece comprises at least three ribs extending radically outwards.

The single piece can be a plate.

A connector is provided at the tail end of the rib, and is configured to securely connect the rib and the outer portion.

The connector is selected from a protrusion, a hook, a convex structure or any other structure that is able to interlock with the outer portion.

The foldable central superelastic alloy component comprises at least two alloy members.

The alloy member can be overlaid atop one another at an angle and can be connected at the overlapping points.

The foldable central superelastic alloy component may comprise at least one connection, and the multiple alloy members are connected via the connection.

The connection may comprise a fixing component or adhesives.

The fixing component can be a plate.

A connector is provided at the tail end of the rib, and is configured to securely connect the rib and the outer portion.

The connector is selected from a protrusion, a hook, a convex structure or any other structure that is able to interlock with the outer portion.

The foldable central superelastic alloy component comprises at least two independent alloy members, and the alloy members are received by the outer portions.

A connector is provided at the tail end of the rib, and is configured to securely connect the rib and the outer portion.

The connector is selected from a protrusion, a hook, a convex structure or any other structure that is able to interlock with the outer portion.

The foldable central superelastic alloy component comprises a single alloy member, and the single alloy member is shaped so as to form all of the ribs.

A connector is provided at the tail end of the rib, and is configured to securely connect the rib and the outer portion.

The connector is selected from a protrusion, a hook, a convex structure or any other structure that is able to interlock with the outer portion.

### BRIEF DESCRIPTION OF THE DRAWINGS

Embodiments of the invention are provided by way of example only, and will be better understood and readily apparent to one of ordinary skill in the art from the following written description and the drawings, in which:

FIG. 1A illustrates a schematic diagram showing a gastric residence article in an expanded configuration in accordance with an example embodiment.

FIG. 1B illustrates a schematic diagram showing the gastric residence article of FIG. 1A in a folded configuration.

FIG. 1C illustrates a schematic diagram showing the gastric residence article of FIG. 1A in a storage configuration.

FIG. 2A illustrates a schematic diagram showing a gastric residence article in an expanded configuration in accordance with another example embodiment.

FIG. 2B illustrates a schematic diagram showing the gastric residence article of FIG. 2A in a folded configuration.

FIG. 3A illustrates a schematic diagram showing a gastric residence article in an expanded configuration in accordance with another example embodiment.

FIG. 3B illustrates a schematic diagram showing the gastric residence article of FIG. 3A in a folded configuration.

FIG. 4A illustrates a schematic diagram showing a gastric residence article in an expanded configuration in accordance with another example embodiment.

FIG. 4B illustrates a transparent section view showing internal elements of the gastric residence article of FIG. 4A.

FIG. 4C illustrates a schematic diagram showing the gastric residence article of FIG. 4A in a folded configuration.

FIG. 4D illustrates a schematic diagram showing alloy members of FIG. 4B.

FIG. 5A illustrates a schematic diagram of a semi-finished foldable central component in accordance with an example embodiment.

FIG. 5B illustrates a schematic diagram of a semi-finished foldable central component in accordance with another example embodiment.

FIG. 5C illustrates a schematic diagram of two foldable central components completed using the semi-finished product of FIG. 5B.

FIG. 6A illustrates a perspective view of an upper part of an outer portion of a gastric residence article in accordance with an example embodiment.

FIG. 6B illustrates a top view of the upper part of FIG. 3A.

FIG. 6C illustrates a side view of the upper part of FIG. 6A.

FIG. 6D illustrates the upper part of FIG. 6A with superelastic alloy wires disposed thereon.

FIG. 6E illustrates a schematic diagram of two foldable central components completed using the upper part of FIG. 6A.

FIG. 7A illustrates a testing jig for conducting tests on the gastric residence article of FIG. 4A.

FIG. 7B illustrates a bar graph showing testing results of a test conducted using the testing jig of FIG. 7A.

FIG. 8A illustrates a schematic diagram showing a foldable central superelastic alloy component comprising a single alloy member.

FIG. 8B illustrates a schematic diagram showing a single alloy member of the foldable central superelastic alloy component in FIG. 8A.

FIG. 9 illustrates a schematic diagram showing a foldable central superelastic alloy component comprising a single piece.

FIG. 10 illustrates a schematic diagram showing a connected alloy member according to an embodiment of the present application.

FIG. 11 illustrates a schematic diagram showing a connected alloy member according to an embodiment of the present application.

FIG. 12 illustrates a schematic diagram showing a connected alloy member according to an embodiment of the present application.

FIG. 13A illustrates a schematic diagram showing alloy members in an unassembled state according to an embodiment of the present application.

FIG. 13B illustrates a schematic diagram showing the alloy members in FIG. 13 in an assembled state.

FIG. 14 illustrates a schematic diagram showing a foldable central superelastic alloy component according to an embodiment of the present application.

### DETAILED DESCRIPTION OF THE EMBODIMENTS

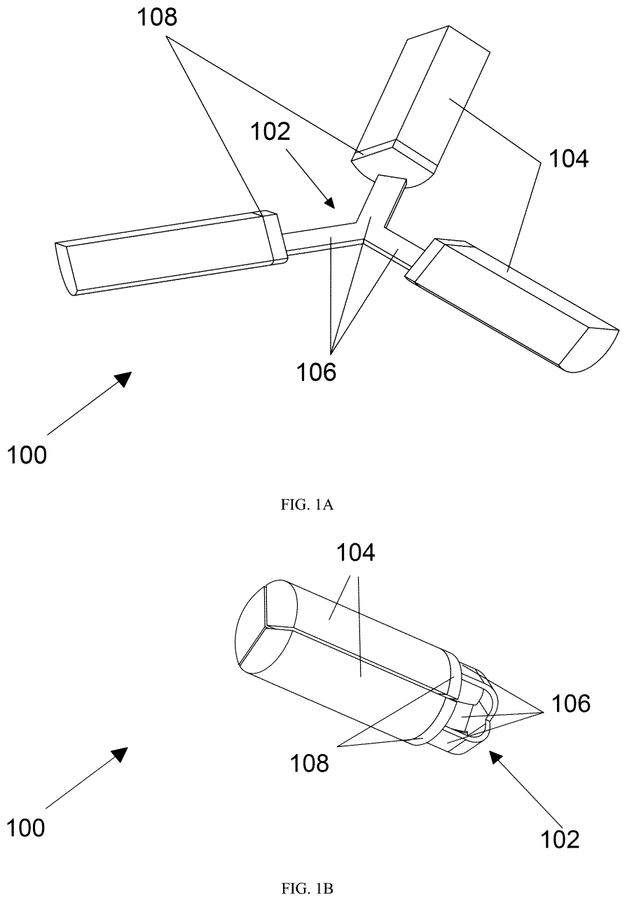

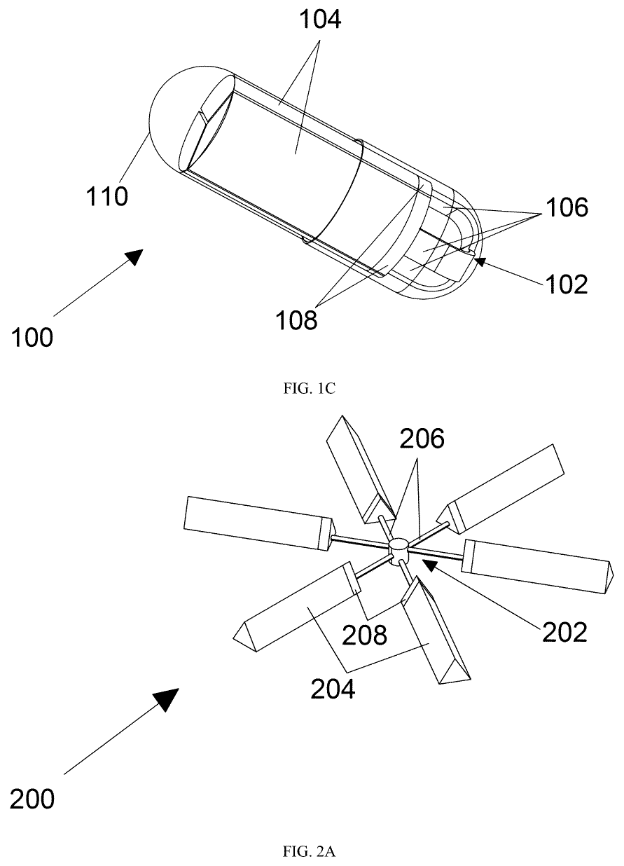

FIG. 1A illustrates a schematic diagram showing a gastric residence article 100 in an expanded configuration in accordance with an example embodiment. FIG. 1B illustrates a schematic diagram showing the gastric residence article of FIG. 1A in a folded configuration. FIG. 1C illustrates a schematic diagram showing the gastric residence article of FIG. 1A in a storage configuration.

The gastric residence article 100 includes a foldable central component 102 and three elongated limb components 104 radially attached to the foldable central component 102. The foldable central component 102 includes three ribs 106 made of at least one superelastic alloy. As shown in FIG. 1A, there are three elongated limb components 104 which correspond to the three ribs 106. The position and longitudinal axes of the elongated limb components 104 correspond to the position and longitudinal axes of the ribs 106 respectively. The elongated limb components 104 have a triangular prism shape that allows the elongated limb components 104 to fit tightly when the gastric residence article 100 is in the folded configuration for easy storage and oral administration, as shown in FIGS. 1B and 1C. The ribs 106 are releasable from a folded first state as shown in FIGS. 1B and 1C to an unfolded second state as shown in FIG. 1A for deploying the elongated limb components 104, resulting in the gastric residence article 100 transforming from the folded configuration to the expanded configuration. The gastric residence article 100 has an opening diameter of at least 2 cm and a folding force of at least 0.5 N in the expanded configuration.

The gastric residence article further includes three linker components 108. Each of the linker components 108 connects one of the elongated limb components 104 to the foldable central component 102. At least one of the elongated limb components 104 is configured to releasably store an active substance or a drug such as a therapeutic agent or a diagnostic agent for gastroretentive drug delivery by releasing the active substance or drug at a predetermined rate.

The linker components 108 are configured to transform from a rigid first state to a disengaged second state to allow the elongated limb components 104 to move independently with respect to the foldable central component 102. The linker components 108 comprise a material that undergoes hydrolysis to transform the linker components 108 from the rigid first state to the disengaged second state. Specifically, the linker components 108 degrades, dissolves, disassociates, or mechanically weakens in a gastric environment, resulting in the gastric residence article 100 losing its rigidity and shape and passing through a gastric cavity of a patient. In one embodiment, the linker components 108 lose its rigidity and become flexible in the disengaged second state to allow bending of the elongated limb components 104 with respect to the foldable central component 102. In another embodiment, the linker components 108 break apart in the disengaged second state to disconnect the elongated limb components 104 from the foldable central component 102.

As shown in FIG. 1C, the gastric residence article 100 further includes a casing 110 that houses the foldable central component 102 and the elongated limb components 104 for easy storage and oral administration. Following oral administration, the casing 112 will degrade, dissolve, disassociate, or mechanically weaken in vivo and the ribs 106 will recoil from the folded first state into the unfolded second state to deploy the elongated limb components 104, causing the gastric residence article 100 to transform from the folded configuration to the expanded configuration for retention of the gastric residence article 100 in a patient's gastric cavity for an extended duration, e.g. 24 hours or more.

In an embodiment, the ribs 106 includes at least one superelastic alloy selected from a group consisting of Nitinol (Ni—Ti), Brass (Cu—Zn), copper-aluminium-nickel (Cu—Al—Ni) alloy, gold-cadmium (Au—Cd) alloy, gold-copper-zinc (Au—Cu—Zn) alloy, indium-thallium (In—Tl) alloy, cobalt-nickel-aluminium (Co—Ni—Al) alloy, cobalt-nickel-gallium (Co—Ni—Ga) alloy, copper-aluminium-beryllium-zirconium (Cu—Al—Be—Zr), copper-aluminium-beryllium-chromium (Cu—Al—Be—Cr) alloy, copper-aluminium-beryllium-gadolinium (Cu—Al—Be—Gd) alloy, copper-aluminium-nickel-hafnium (Cu—Al—Ni—Hf) alloy, copper-tin (Cu—Sn) alloy, copper-zinc-silicon (Cu—Zn—Si) alloy, copper-zinc-aluminium (Cu—Zn—Al) alloy, copper-zinc-silicon (Cu—Zn—Sn) alloy, iron-manganese-silicon (Fe—Mn—Si) alloy, iron-platinum (Fe—Pt) alloy, manganese-copper (Mn—Cu) alloy, nickel-iron-gallium (Ni—Fe—Ga) alloy, (Ni—Ti—Hf) alloy, nickel-titanium-palladium (Ni—Ti—Pd) alloy, nickel-manganese-gallium (Ni—Mn—Ga) alloy, nickel-manganese-gallium-copper (Ni—Mn—Ga—Cu) alloy, nickel-manganese-gallium-cobalt (Ni—Mn—Ga—Co) alloy, titanium-niobium (Ti—Nb) alloy.

The ribs 106 can also be made of a shape memory alloy selected from a group consisting of Ni—Ti, Ni—Ti—Hf, Ni—Ti—Pd, Ni—Fe—Ga, Ni—Mn—Ga, Ni—Mn—Ga—Cu, Ni—Mn—Ga—Co, Ag—Cd. Co—Ni—Al, Co—Ni—Ga, Cu—Al—Be—X (X: Zr, B, Cr, Gd), Cu—Al—Ni, Cu—Al—Ni—Hf, Cu—Sn, Cu—Zn, Cu—Zn—X (X=Si, Al, Sn), Fe—Mn—Si, Fe—Pt, Mn—Cu, Ti—N.

Shape memory alloys belong to the family of superelastic alloys. A suitable shape memory alloy for the ribs 106 has an austenite transformation finish temperature (Af) slightly below normal body temperature. At temperatures below the Af (e.g. 30° C.), shape memory alloys are in martensite phase and can be malleable. However, when the shape memory alloys are heated to a temperature above the Af, they become superelastic and will revert to their initial unfolded state. During production and storage, the shape memory alloys are in the martensite phase and thus, the gastric residence article 100 can retain its folded configuration without a restraining structure such as the casing 110. Following oral administration of the gastric residence article 100, the temperature of the shape memory alloy increases due to the internal heat of a patient's body. This causes the shape memory alloy to go through the transformation from martensite phase to austenite phase, thereby allowing the elongated limb components 104 to expand for retention of the gastric residence article 100 in the patient's gastric cavity. The opening diameter is the diameter or diagonal of a largest cross-section of the gastric residence article in the expanded configuration of the gastric residence article.

The gastric residence article 100 has a high creep resistance for the self-expanding mechanism to spring back to its expanded configuration after long term storage. In an embodiment, the gastric residence article in the expanded configuration may have an opening diameter of at least 2 cm, for example, 2 cm, 2.1 cm or 2.2 cm in the expanded configuration after being stored at a storage temperature for a storage time.

In an embodiment, after the gastric residence article 100 is manufactured, the ribs of the gastric residence article 100 may be fully unfolded. In the fully unfolded configuration, all of the ribs are located in a common plane, which can be defined by an unfolding angle of 0°. After being stored at a storage temperature for a storage time, the unfolding angle is no more than 45°, for example, 5°, 6″, 7°, 8°, 9°, 10°, 15°, 20°, 25°, 30°, 35°, 40° or 45°.

The storage time refers to the period before use after the completion of the manufacturing of the gastric residence article 100. The disclosed gastric residence article 100 can still have good unfolding ability after an extended storage time, since the ribs include the at least one superelastic alloy. That is, the gastric residence article 100 has an extended shelf life. The storage time of the gastric residence article 100 according to the present disclosure may be, for example, at least 6 months, at least 9 months, at least 10 months, at least 11 months, at least 1 year or at least 2 year.

The storage temperature is below, for example, 20° C. 21° C. 22° C. 23° C. 24° C. 25° C., 26° C., 27° C., 28° C., 29° C., 30° C., 31° C., 32° C., 33° C., 34° C., 35° C., 36° C., 37° C., 38° C., 39° C., 40° C. 41° C., 42° C., 43° C., 44° C. or 45° C.

FIG. 2A illustrates a schematic diagram showing a gastric residence article 200 in an expanded configuration in accordance with another example embodiment. FIG. 2B illustrates a schematic diagram showing the gastric residence article 200 of FIG. 2A in a folded configuration.

The gastric residence article 200 includes a foldable central component 202 and six elongated limb components 204 radially attached to the foldable central component 202. The foldable central component 202 includes six ribs 206 made using superelastic alloy wires. The gastric residence article 200 further includes six linker components 208 for connecting each of the elongated limb components 204 to the foldable central component 202. At least one of the elongated limb components 204 is configured to store an active substance or a drug such as a therapeutic agent or a diagnostic agent for gastroretentive drug delivery. The linker components 208 are configured to transform from a rigid first state to a disengaged second state to allow the elongated limb components 204 to move independently with respect to the foldable central component 202.

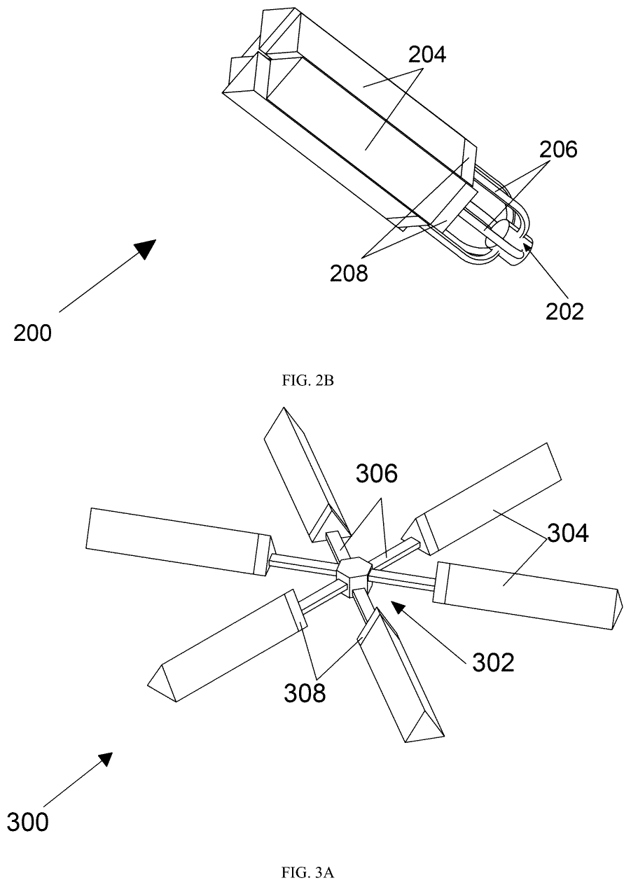

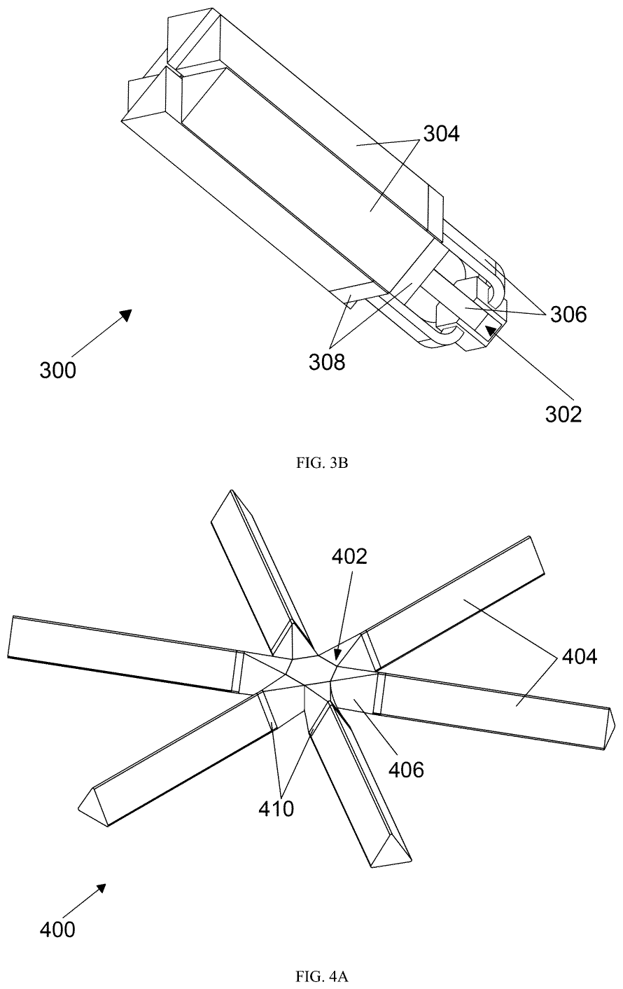

FIG. 3A illustrates a schematic diagram showing a gastric residence article 300 in an expanded configuration in accordance with another example embodiment. FIG. 3B illustrates a schematic diagram showing the gastric residence article 300 of FIG. 3A in a folded configuration.

The gastric residence article 300 includes a foldable central component 302 and six elongated limb components 304 radially attached to the foldable central component 302. The foldable central component 302 includes six ribs 306 made using superelastic alloy ribbons. The gastric residence article 300 further includes six linker components 308 for connecting each of the elongated limb components 304 to the foldable central component 302. At least one of the elongated limb components 304 is configured to store an active substance or a drug such as a therapeutic agent or a diagnostic agent for gastroretentive drug delivery. The linker components 308 are configured to transform from a rigid first state to a disengaged second state to allow the elongated limb components 304 to move independently with respect to the foldable central component 302. Multiple ribs 306 will bear the force in the human body thus distributing the force over multiple ribs 306.

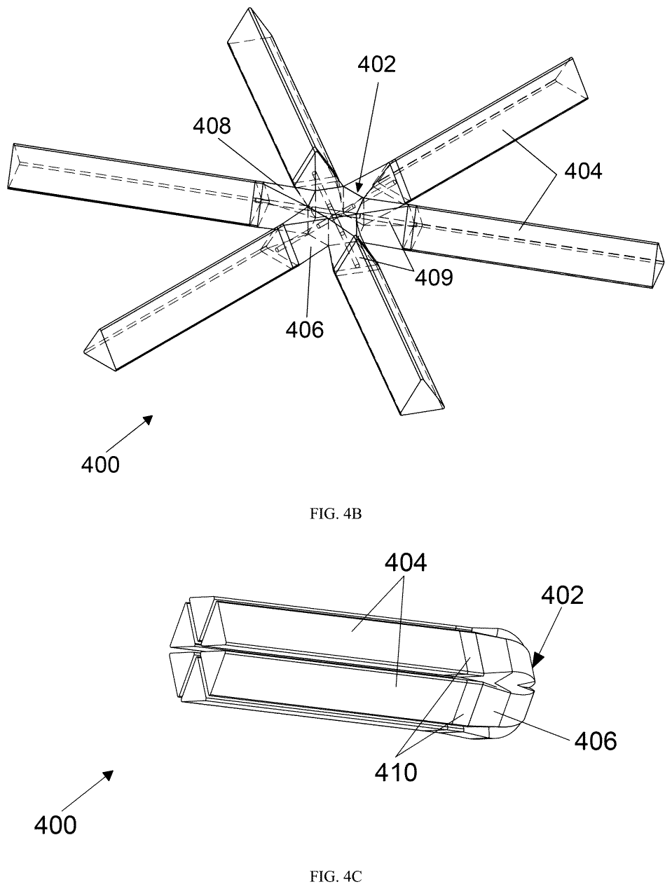

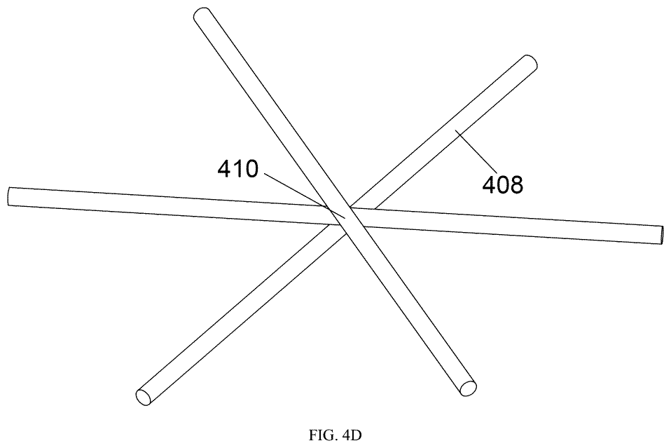

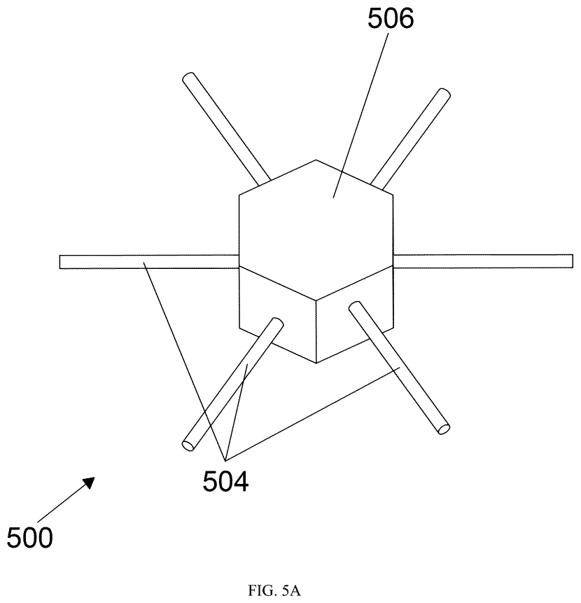

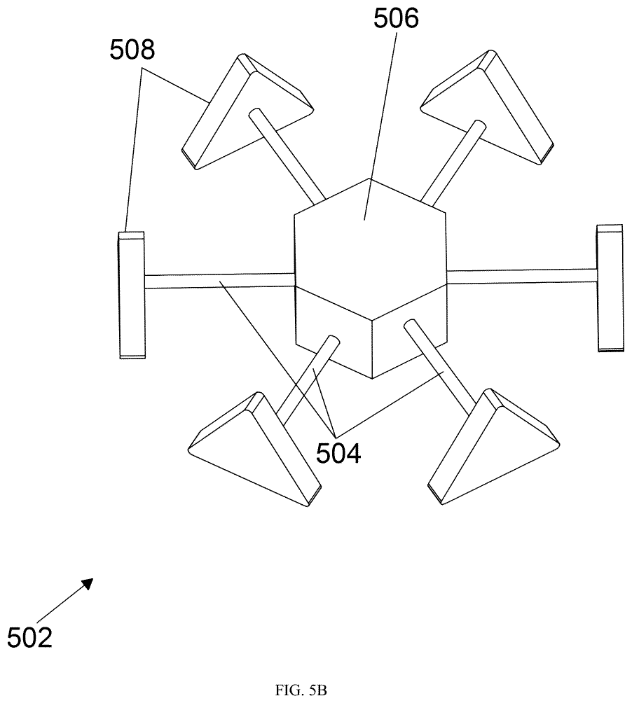

FIG. 4A illustrates a schematic diagram showing a gastric residence article 400 in an expanded configuration in accordance with an example embodiment. FIG. 4B illustrates a transparent section view showing internal elements of the gastric residence article 400 of FIG. 4A. FIG. 4C illustrates a schematic diagram showing the gastric residence article 400 of FIG. 4A in a folded configuration.

The gastric residence article 400 includes a foldable central component 402 and a plurality of elongated limb components 404 radially attached to the foldable central component 402. The foldable central component 402 includes an outer portion 406 and a plurality of ribs 408 made of superelastic alloy wires securely received within the outer portion 406, as shown in FIG. 4B. The gastric residence article 400 further includes a plurality of linker components 410 for connecting each of the elongated limb components 404 to the outer portion 406. The foldable central component 402 includes multiple alloy members 401, these alloy members 401 are superelastic alloy wires, which are separate parts and independent from each other when assembled, as shown in FIG. 4D.

The outer portion 406 includes a soft foldable material and is made of at least one selected from a group consisting of polypropylene, polystyrene, polyvinyl chloride, synthetic rubber, phenol formaldehyde resin (or Bakelite), neoprene, nylon, polyacrylonitrile, PVB, silicone, acrylonitrile butadiene styrene, high density polyethylene, polycarbonate, nylon, acrylic, polyethylene terephthalate, polybutylene terephthalate, acetal, polyimide, polyurethane, and epoxy.

The main function of the outer portion 406 is to adhere to the elongated limb components 404. In an embodiment, at least 80% of the folding force is attributed to the foldable central component 402 and less than 20% of the folding force is attributed to the outer portion 406. Thus, many materials can be used to make the outer portion 406 as the properties such as the creep resistance, flexural modulus and degradation time of the materials are not the main considerations when selecting the materials for the outer portion 406.

In an embodiment, the outer portion 406 is formed by applying the polymer over the superelastic alloy ribs 408 via injection molding or permanent mold casting. It should be noted that the ribs 408 are not limited to only the shape of a wire and that the ribs 408 can include other shapes such as the shape of a ribbon or a bar. The outer portion 406 can be formed over the ribs 408 in any of these shapes such that the ribs 408 are securely received within the outer portion 406.

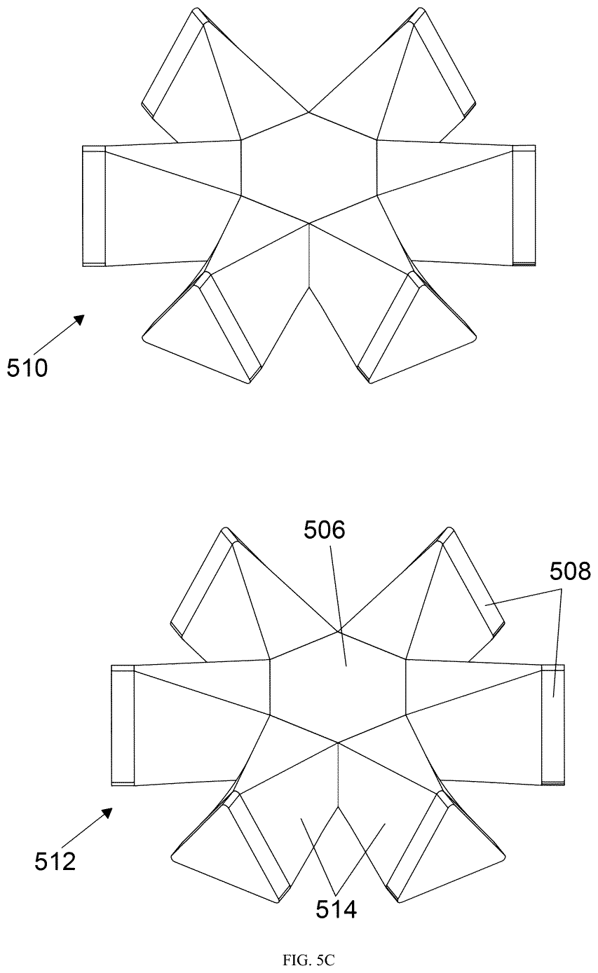

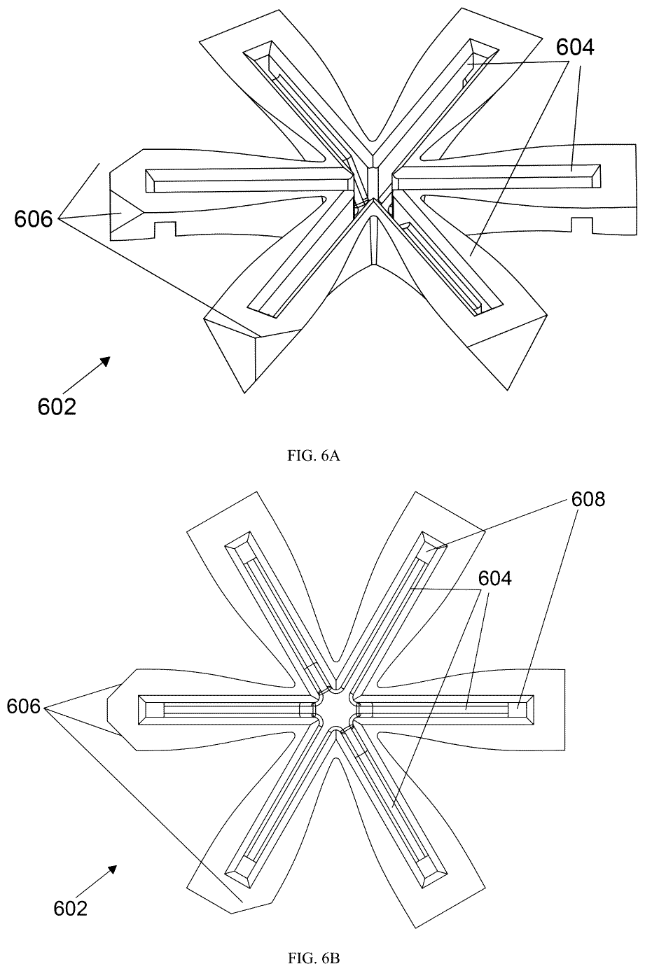

FIG. 5A illustrates a schematic diagram of a semi-finished foldable central component 500 in accordance with an example embodiment. FIG. 5B illustrates a schematic diagram of a semi-finished foldable central component 502 in accordance with another example embodiment. Three superelastic alloy wires 504 made of nitinol are held in a first mold in an overlapping manner. A thermoplastic polymer is injected into the first mold to form an inner part, represented as molded part 506 in FIGS. 5A and 5B, at the overlapped region of the superelastic alloy wires 504 to secure the relative positions of the superelastic alloy wires 504. The first mold can also be made such that the thermoplastic polymer forms molded parts 508 at the distal ends of the superelastic alloy wires 504, as shown in FIG. 5B. Next, the semi-finished product is transferred to a second mold and the thermoplastic polymer is injected into the second mold to complete the foldable central component.

FIG. 5C illustrates a schematic diagram of two foldable central components 510, 512 completed using the semi-finished product of FIG. 5B. The foldable central component 510 is made of the same polymer while the foldable central component 512 is made of the different polymers. As an example, in the foldable central component 512, a first polymer that has high hardness can be used to form the molded part 506 at the overlapped region of the superelastic alloy wires 504 to fix the relative positions of the superelastic alloy wires 504, a second polymer can be used to form the molded parts 508 at the distal ends of the superelastic alloy wires 504 to form strong connections with the linker components and a third polymer that has low hardness can be used to form molded part 514 that cover the length of the superelastic alloy wires 504 to allow bending of the superelastic alloy wires 504.

In an embodiment, the thermoplastic polymer is first injected into the first mold to form the molded part 506 and the superelastic alloy wires 504 are subsequently placed into the molded part 506 to be secured in place.

In an embodiment, the molded parts 506, 508, 514 can be made by permanent mold casting instead of injection molding. The thermoplastic polymer is poured into the first mold or second mold and is allowed to harden by processes such as cooling or curing.

It should be noted that the ribs are not limited to only the shape of a wire as shown in FIGS. 5A and 5B and that the ribs can include other shapes such as the shape of a ribbon or a bar.

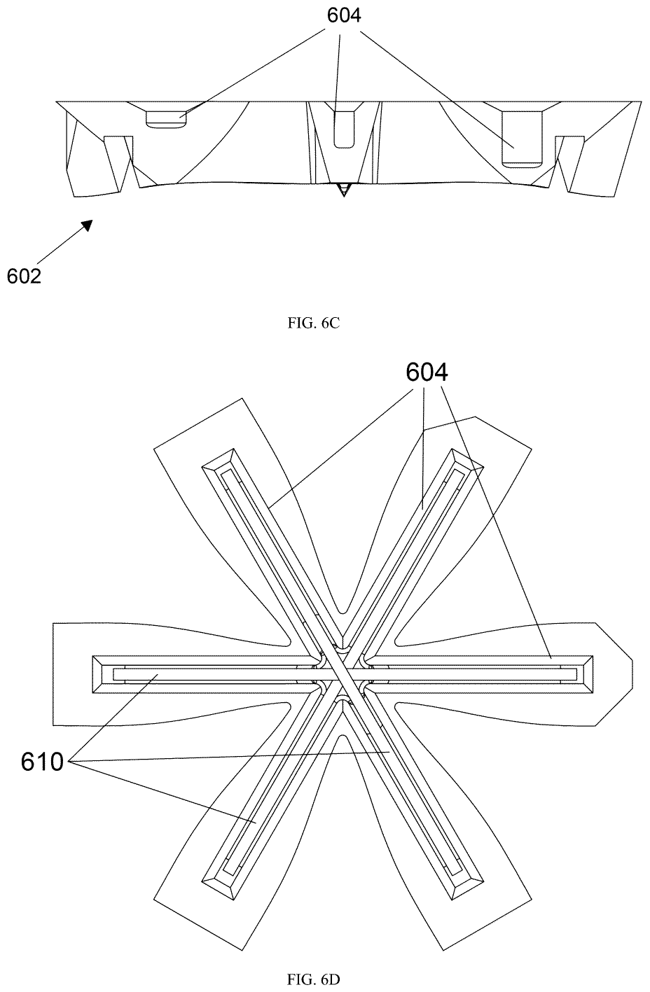

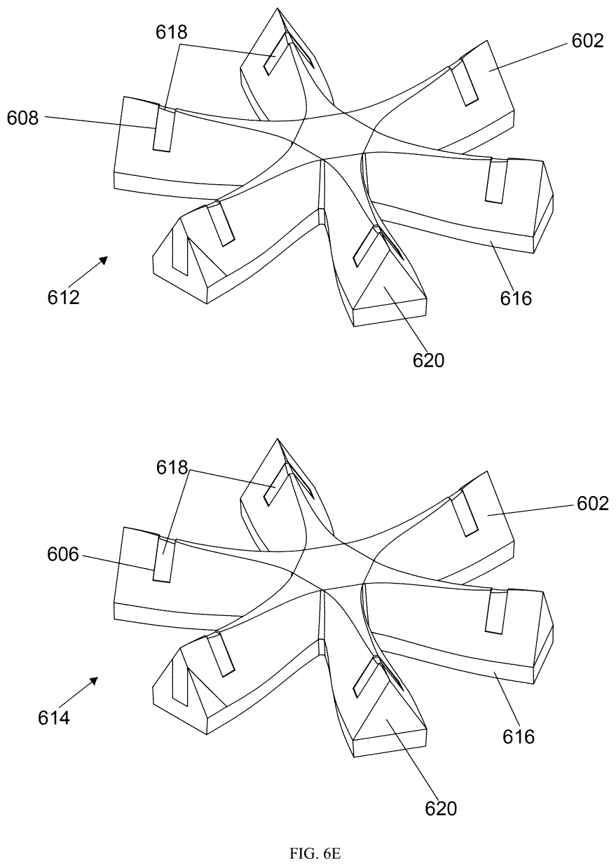

FIG. 6A illustrates a perspective view of an upper part 602 of an outer portion of a gastric residence article in accordance with an example embodiment. FIG. 6B illustrates a top view of the upper part 602 of FIG. 6A. FIG. 6C illustrates a side view of the upper part 602 of FIG. 6A.

The upper part 602 includes slots 604 for containing the internal superelastic alloy, which in this case are three superelastic alloy wires. As can be seen in FIG. 6C, the slots 604 have different depths to ensure that the superelastic alloy wires are levelled at the respective depths. Reference markers, such as notches 606 shown in FIGS. 6A and 6B, are made to guide the sequence in disposing the superelastic alloy wires in the slots 604 of the upper part 602. The upper part 602 further includes openings 608 that will allow for a stronger bond with the lower part when it is fused with the upper part.

FIG. 6D illustrates the upper part 602 of FIG. 6A with superelastic alloy wires disposed thereon. Three superelastic alloy wires 610 are disposed into the slots 604 of the upper part 602. In the preparation process of the upper part 602 shown in FIG. 6D, three superelastic alloy wires 610 are sequentially placed in the upper part 602, and the three superelastic alloy wires 610 overlap with each other at the slots 604. In an embodiment, a midpoint of each of the three superelastic alloy wires 610 is located at the overlap position of the three superelastic alloy wires 610. Thereafter, polymer is injected into the slots 604 of the upper part 602 to encase the superelastic alloy wires 610 within the outer portion, to form the foldable central component 612.

FIG. 6E illustrates a schematic diagram of two foldable central components 612, 614 completed using the upper part 602 of FIG. 6A. Each of the foldable central components 612, 614 includes the upper part 602 and the lower part 616 connected to each other. The upper part 602 and the lower part 616 of the foldable central component 612 are made of the same polymer while the upper part 602 and the lower part 616 of the foldable central component 614 are made of different polymers.

In an embodiment, the upper and lower parts 602, 616 are made by injection molding or permanent mold casting. A thermoplastic polymer is injected into a first mold to form the upper part 602. The superelastic alloy wires 610 are disposed into the slots 604 formed in the upper part 602. Next, the upper part 602 with the superelastic alloy wires 610 disposed within the slots 604 is placed in a second mold and the thermoplastic polymer is injected into the second mold to form the lower part 616 via overmolding, thereby forming the foldable central component 612, 614.

In an embodiment, the slots for containing the superelastic alloy wires can be formed in the lower part 616 instead of in the upper part 602. In another embodiment, the slots for containing the superelastic alloy wires 610 can be formed in both the upper part 602 and lower part 616.

In an embodiment, the lower part 616 made via overmolding with the second mold extends to cover distal tips 620 of the upper part 602 such that the linker components of the gastric residence article connect to the lower part 616.

In an embodiment, the linker components and elongated limb components of the gastric residence article are manufactured via hot melt extrusion. The materials are fed into a hot melt extruder, and a filament is extruded at the desired cross-sectional shape and cut into a suitable length to form the linker components and elongated limb components.

In an embodiment, the linker components and elongated limb components are manufactured via injection molding. The materials are first blended to form solid granules. This can be achieved by performing hot melt extrusion on the materials to produce extruded filaments and subsequently palletizing the extruded filament to form the solid granules. The solid granules can also be made by dissolving the materials in a suitable solvent to form a solution and pouring the solution onto a flat surface to form a sheet which is then broken down to form solid granules. The solid granules are then fed into an injection molding equipment to form the linker components and loadable elongated limb components.

In an embodiment, the linker components and elongated limb components are manufactured via permanent mold casting. The materials are first mixed to form a casting solution which is then poured into a mold in the shape of the linker components and elongated limb components and allowed to harden by processes such as cooling or curing to form the linker components and elongated limb components.

In an embodiment, the foldable central component can be connected to the linker components and elongated limb components via thermal bonding. The foldable central component and the elongated limb components connected to the linker components are placed into a mold and heat is applied at the boundary between the foldable central component and the linker components to fuse them together. A push force can be applied to the distal parts of the elongated limb components to press the linker components against the foldable central component for a stronger bond. The heat can be applied using laser, heating elements within the mold, etc. It should be noted that the foldable central component and the linker components can be connected via other methods, e.g. solvent bonding and using an adhesive.

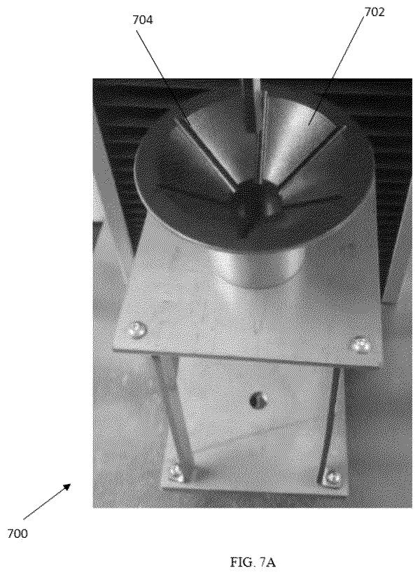

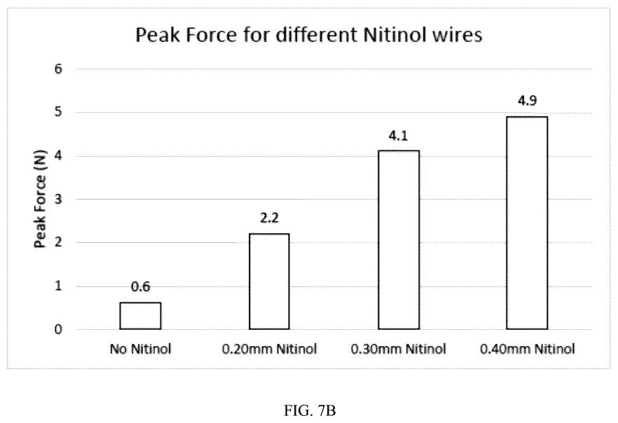

FIG. 7A illustrates a testing jig 700 for conducting tests on the gastric residence article 400 of FIG. 4A. FIG. 7B illustrates a bar graph showing testing results of a test conducted using the testing jig 700 of FIG. 7A. The testing jig 700 includes a funnel 702 with a bottom opening of 20 mm to simulate the pyloric opening of the stomach in the body of a patient. The testing jig 700 is used to determine the peak force required to push the gastric residence article 400 through the bottom opening. Specifically, the gastric residence article 400 is placed into the funnel 702 with each elongated limb component of the gastric residence article 400 placed in the grooves 704 of the funnel 702. A probe is then used to press the article 400 through the funnel 702 and the peak force is recorded.

A sideway force test simulates the gastric residence article 400 leaving the pyloric opening sideways and a planar force test simulates the gastric residence article 400 leaving the pyloric opening planarly. The four gastric residence articles 400 tested include no nitinol wire, and nitinol wires with cross-sectional area 0.20 mm, 0.30 mm and 0.40 mm, and the peak force required to push the gastric residence articles 400 through the bottom opening are 0.6 N, 2.2 N, 4.1 N and 4.9 N respectively.

Embodiments of the present invention provide a gastric residence article 100 for oral administration. The gastric residence article 100 includes a plurality of ribs 106 that are made of at least one superelastic alloy which generally have a high flexural modulus. Thus, superelastic alloy ribs 106 that are relatively small can achieve the flexural force required to retain the gastric residence article 100 within the stomach. This may advantageously allow for flexibility in the design of the gastric residence article 100.

For example, the gastric residence article 100 can be in made in different sizes and shape using a similar superelastic alloy as only the length of the ribs 106 needs to be changed. Gastric residence articles 100 that are small allows for easy swallowing and can be used to treat small animals or patients with small gastrointestinal tracts such as children. The superelastic alloy ribs 106 also allow for easy tuning of the flexural force since only the cross sectional area of the bending points of the superelastic alloy ribs 106 needs to be adjusted. Thus, gastric residence articles 100 of the same size and shape can have different capabilities for retention in the stomach due to using superelastic alloy ribs 106 of different cross-sectional areas which results in the change in the flexural force of the superelastic alloy ribs 106.

Some variations of the foldable central superelastic alloy component are provided as follows.

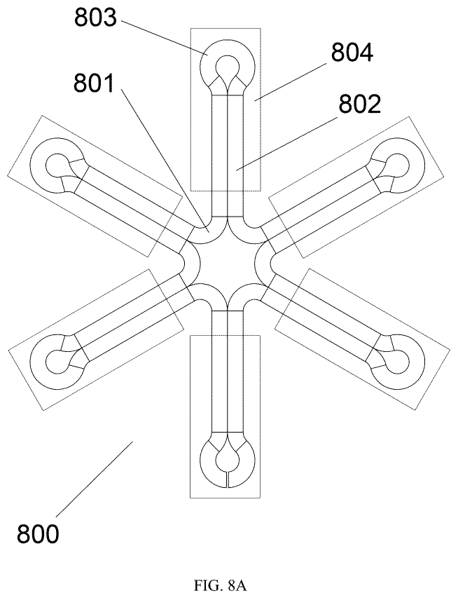

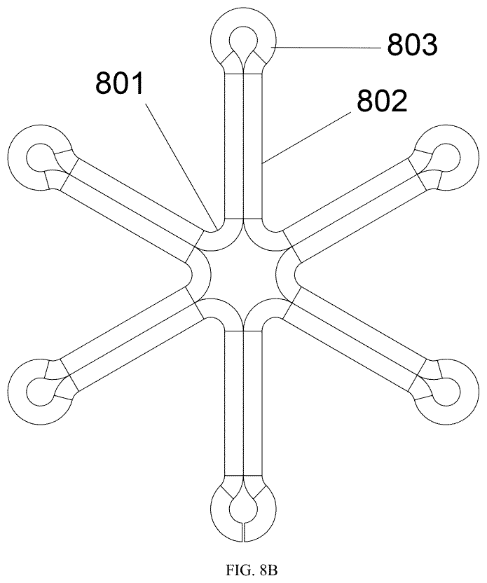

FIG. 8A shows a variation of the foldable central superelastic alloy component 800. In this variation, the foldable central superelastic alloy component comprises a single alloy member 801, and the single alloy member 801 is shaped so as to form all of the ribs 802. The single alloy member 801 is a single superelastic alloy wire. The ribs 802 are securely received within outer portions respectively, A connector 803 is provided at the tail end of the rib 802, and is configured to securely connect the rib 802 and the outer portion 804, the connector 803 is a protrusion. In another embodiment, the connector 803 can have other shapes, for example a hook, a convex structure or any other structure that is able to interlock with the outer portion.

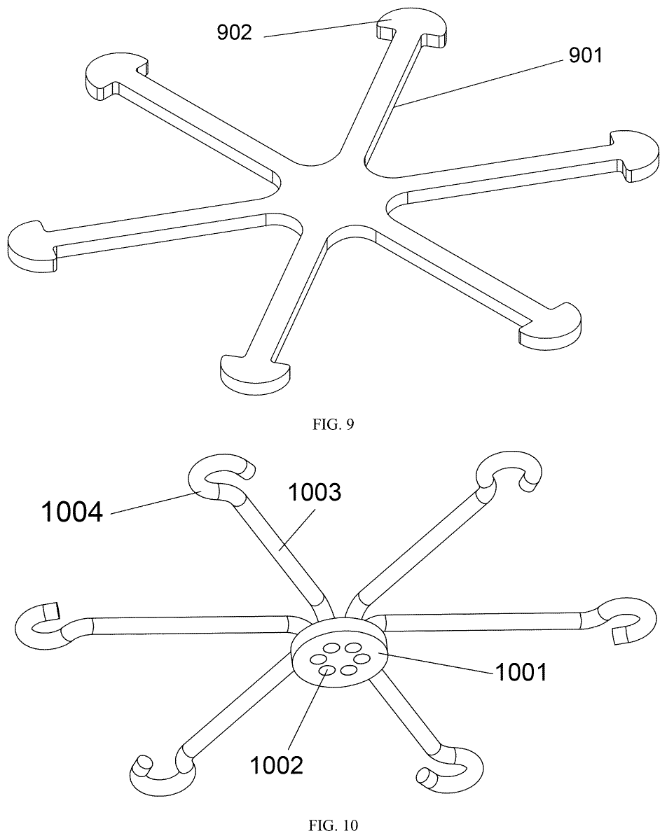

FIG. 9 shows a variation of the foldable central superelastic alloy component. The foldable central superelastic alloy component is a single piece and the single piece comprise six ribs 901 extending radically outwards. A connector 902 is provided at the tail end of the rib 901, and is configured to securely connect the rib 901 and the outer portion, the connector 902 is a protrusion. In another embodiment, the connector 902 can have other shapes, for example a hook, a convex structure or any other structure that is able to interlock with the outer portion.

The foldable central superelastic alloy component comprises at least two connected alloy members. The alloy member can be overlaid atop one another at an angle and can be connected at the overlapping points. For example, as shown in the FIG. 12, the foldable central superelastic alloy component comprises six connected alloy members 1203 and one connection 1201, and the alloy members 1203 are connected together via the connection 1201. In the FIG. 12, the connection 1201 is a cylinder 1201 with fixing slots or holes 1202 uniformly distributed on a side surface of the cylinder 1201. The alloy member 1203 is the superelastic alloy wire. One end of the superelastic alloy wire is inserted into the corresponding fixing slot or hole 1202, so that positioning and installation of alloy members 1203 are convenient, and then the multiple alloy members 1203 are fixed in the fixing slot or hole 1202 by welding or adhesive, so as to form the foldable central superelastic alloy component. The other end of the superelastic alloy wire is oriented in the radial direction of the cylinder. The connection 1201 can be made of metal, alloy, or polymer, etc.

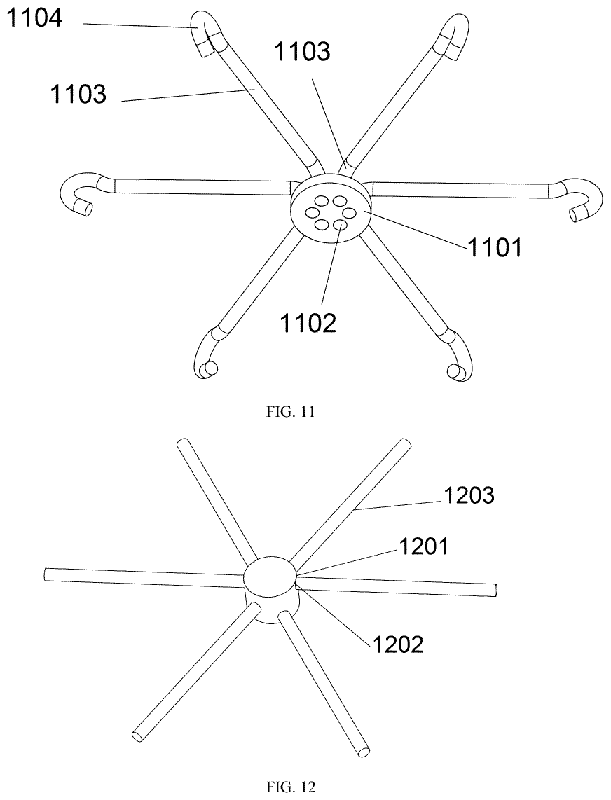

In another embodiment, as shown in the FIGS. 10 and 11, the connection 1001, 1101 is a center fixing plate with fixing slots or holes 1002, 1102 uniformly distributed on a top surface of the center fixing plate is provided. One end of the superelastic alloy wire 1003, 1103 is inserted into the corresponding fixing slot or hole 1002, 1102, and is fixed in the fixing slot or hole 1002, 1102 by welding or adhesive, so as to form the foldable central superelastic alloy component. The other end of the superelastic alloy wire 1003, 1103 is oriented in the radial direction of the center fixing plate. Optionally, the foldable central superelastic alloy component can be configured to be connected to the outer portion. A connector 1004, 1104 is provided at the tail end of the superelastic alloy wire 1003, 1103, and is configured to securely connect the superelastic alloy wire 1003, 1103 and the outer portion, the connector 1004, 1104 is hooked. The connector 1004, 1104 is bent in different directions. Optionally, the center fixing plate may be circular or annular. Optionally, the hook-like end of the superelastic alloy wire can be bent in a direction parallel with, or perpendicular to a plane in which the center fixing plate is located.

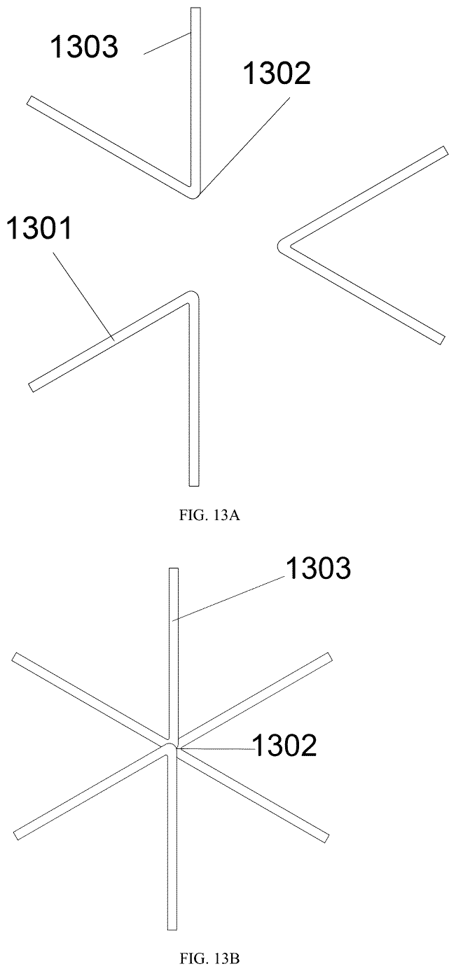

In another embodiment, as shown in the FIG. 13A, each alloy member 1301 has a bending portion 1302, two ends of the bending portion 1302 of the alloy member 1301 extend to form two ribs 1303 respectively. FIG. 13B shows that the alloy members from FIG. 13A overlaid atop one another at the bending portion 1302 and are connected at the bending portion 1302.

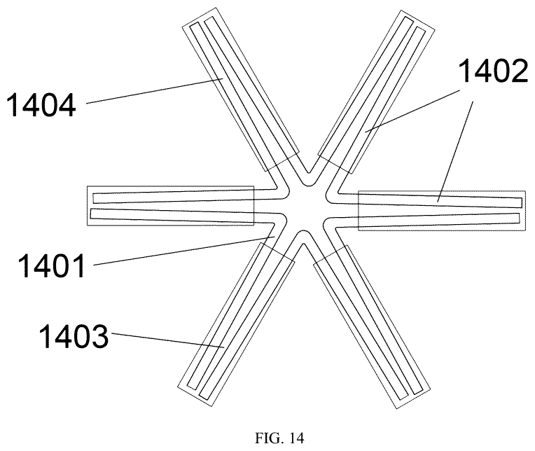

In another embodiment, as shown in the FIG. 14, the foldable central superelastic alloy component comprises multiple alloy members 1401, each of the multiple alloy members is bent to form two angled legs 1402. Each rib 1404 is composed of two adjacent angled legs 1402 which are held by the outer portions 1403.

The superelastic alloy component can be produced by stamping, EDM wire cutting, laser cutting or 3D metal printing process.

Assembly process of the superelastic alloy component including separate parts can be as follows: for the superelastic alloy component shown in FIG. 4D in which no central support is provided, the wires can be directly welded to each other to form the superelastic alloy component; for the superelastic alloy component shown in FIGS. 10 to 11, forming fixing slots or holes in the fixing plate, inserting the wires into the slots or holes, and then fixing the wires to the plate by welding or adhesive to form the superelastic alloy component.

### EXAMPLE 1—Production of Central Foldable Component

1. The central foldable component 614 in FIG. 6E was produced is as follows:
2. Cut 0.30 mm diameter nitinol wires to 17 mm in length.
3. The upper part 602 was produced by injecting molding PC-3575A from Lubrizol into a first mold.
4. The cut 0.30 mm diameter nitinol wires were then carefully placed into the upper part 602.
5. The entire piece with the nitinol wires were then placed into a second mold and PC-3575A from Lubrizol was injected to form part 614.

In step 2, different diameter nitinol wires can be used such as for example 0.10 mm, 0.20 mm, or 040 mm, etc.

### EXAMPLE 2—Production of Gastric Residence Article

1. A gastric residence article using the central foldable component 614 in was produced is as follows:
2. 99 g of HPMC-AS-HG from Shin-Etsu, 99 g of PCL, and 2 g of silica were blended until the mixture was homogenous. The homogenous powder was then loaded into the feeder of a Pharma 11 Twin-Screw Extruder from Thermo Scientific with a customized triangular die head to achieve the triangular extrusion. After extrusion, the triangular filament was then cut to 3 mm segments to form the linker;
3. 99 g of Barium Sulfate, 99 g of PCL, and 2 g of silica were blended until the mixture was homogenous. The homogenous powder was then loaded into the feeder of a Pharma 11 Twin-Screw Extruder from Thermo Scientific with a customized triangular die head to achieve the triangular extrusion. After extrusion, the triangular filament was then cut to 10 mm segments to form the elongated limb component with no active substance;
4. The central foldable component 614, linkers and elongate limb component were then loaded into a customized mold and the different components were heat fused together via a laser to form the gastric residence article;

The gastric residence article was then folded and placed into a size 0 capsule.

### EXAMPLE 3—Animal Study for Retention Duration

Two types of central foldable components were produced using the method of example 1. One type was prepared using 0.30 mm diameter nitinol wire while the other was prepared without the use of any nitinol wire. Three of each of the central foldable components were then made into gastric residence articles as per the method in example 2. The samples prepared are shown the table below.

Female Panama pigs with weights of between 30 kg and 45 kg were then administered one device each. 10 minutes after administration and every day following, fluoroscopy was performed on the pigs to determine if the gastric residence article was still in the stomach. On the day at which the gastric residence article is no long in the stomach was recorded as the retention period. The results of the retention period for the devices are shown in the table below.

| Device | 1 | 2 |
|---|---|---|
| Superelastic alloy | None | 0.30 mm |
| component | | nitinol wires |
| Central region | PC-3575A | PC-3575A |
| Retention period (d) | 1.3 ± 0.5 | 5.3 ± 0.9 |

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

What is claimed is:

1. A gastric residence article for oral administration comprising:
a foldable central superelastic alloy component;
a plurality of elongated limb components loadable with an active substance, the active substance being a therapeutic agent or a diagnostic agent,
wherein the plurality of elongated limb components are each connected to the foldable central superelastic alloy component via a linker component,
wherein the foldable central superelastic alloy component undergoes elastic deformation when the gastric residence article is in a folded configuration and recoils when the gastric residence article assumes an expanded configuration, and
wherein said linker component degrades, dissolves, disassociates, or mechanically weakens, thereby resulting in loss of a shape of the gastric residence article in the expanded configuration.

2. The gastric residence article as claimed in claim 1, wherein the expanded configuration of the gastric residence article has a folding force of at least 0.5 N.

3. The gastric residence article as claimed in claim 1, wherein the expanded configuration of the gastric residence article has an opening diameter of at least 2 cm.

4. The gastric residence article as claimed in claim 1, wherein the gastric residence article is configured to retain the shape in the expanded configuration for at least 24 hours.

5. The gastric residence article as claimed in claim 1, wherein the foldable central superelastic alloy component comprises at least one selected from a group consisting of Nitinol (Ni—Ti), Brass (Cu—Zn), copper-aluminium-nickel (Cu—Al—Ni) alloy, gold-cadmium (Au—Cd) alloy, gold-copper-zinc (Au—Cu—Zn) alloy, indium-thallium (In—Tl) alloy, cobalt-nickel-aluminium (Co—Ni—Al) alloy, cobalt-nickel-gallium (Co—Ni—Ga) alloy, copper-aluminium-beryllium-zirconium (Cu—Al—Be—Zr), copper-aluminium-beryllium-chromium (Cu—Al—Be—Cr) alloy, copper-aluminium-beryllium-gadolinium (Cu—Al—Be—Gd) alloy, copper-aluminium-nickel-hafnium (Cu—Al—Ni—Hf) alloy, copper-tin (Cu—Sn) alloy, copper-zinc-silicon (Cu—Zn—Si) alloy, copper-zinc-aluminium (Cu—Zn—Al) alloy, copper-zinc-silicon (Cu—Zn—Sn) alloy, iron-manganese-silicon (Fe—Mn—Si) alloy, iron-platinum (Fe—Pt) alloy, manganese-copper (Mn—Cu) alloy, nickel-iron-gallium (Ni—Fe—Ga) alloy, (Ni—Ti—Hf) alloy, nickel-titanium-palladium (Ni—Ti—Pd) alloy, nickel-manganese-gallium (Ni—Mn—Ga) alloy, nickel-manganese-gallium-copper (Ni—Mn—Ga—Cu) alloy, nickel-manganese-gallium-cobalt (Ni—Mn—Ga—Co) alloy, titanium-niobium (Ti—Nb) alloy.

6. The gastric residence article as claimed in claim 1, wherein the foldable central superelastic alloy component is securely received within an outer portion.

7. The gastric residence article as claimed in claim 6, wherein at least 80% of the folding force is attributed to the foldable central superelastic alloy component.

8. The gastric residence article as claimed in claim 6, wherein the linker component is coupled to the outer portion.

9. The gastric residence article as claimed in claim 6, wherein the outer portion is made of at least one selected from a group consisting of polypropylene, polystyrene, polyvinyl chloride, synthetic rubber, phenol formaldehyde resin (or Bakelite), neoprene, nylon, polyacrylonitrile, PVB, silicone, acrylonitrile butadiene styrene, high density polyethylene, polycarbonate, nylon, acrylic, polyethylene terephthalate, polybutylene terephthalate, acetal, polyimide, polyurethane, and epoxy.

10. The gastric residence article as claimed in claim 1 further comprising a casing for housing the gastric residence article in the folded configuration, wherein the casing is configured to degrades, dissolves, disassociates, or mechanically weakens to allow the gastric residence article to recoil and assume the expanded configuration.

11. A gastric residence article for oral administration comprising:
a foldable central shape memory alloy component;
a plurality of elongated limb components loadable with an active substance, the active substance being a therapeutic agent or a diagnostic agent,
wherein the plurality of elongated limb components are each connected to the foldable central shape memory alloy component via a linker component,
wherein the foldable central shape memory alloy component undergoes a phase transformation in its crystal structure from the martensite phase to the austenite phase, thereby transforming the article from a folded configuration to an expanded configuration, and wherein said linker component degrades, dissolves, disassociates, or mechanically weakens, thereby resulting in loss of a shape of the gastric residence article in the expanded configuration.

12. The gastric residence article as claimed in claim 11, wherein the expanded configuration of the gastric residence article has a folding force of at least about 0.5 N.

13. The gastric residence article as claimed in claim 11, wherein the expanded configuration of the gastric residence article has an opening diameter of at least 2 cm.

14. The gastric residence article as claimed in claim 11, wherein the gastric residence article is configured to retain the shape in the expanded configuration for at least 24 hours.

15. The gastric residence article as claimed in claim 11, wherein the foldable central shape memory alloy component comprises at least one selected from a group consisting of Ni—Ti, Ni—Ti—Hf, Ni—Ti—Pd, Ni—Fe—Ga, Ni—Mn—Ga, Ni—Mn—Ga—Cu, Ni—Mn—Ga—Co, Ag—Cd, Co—Ni—Al, Co—Ni—Ga, Cu—Al—Be—X (X: Zr, B, Cr, Gd), Cu—Al—Ni, Cu—Al—Ni—Hf, Cu—Sn, Cu—Zn, Cu—Zn—X (X=Si, Al, Sn), Fe—Mn—Si, Fe—Pt, Mn—Cu, Ti—Nb.

16. The gastric residence article as claimed in claim 11, wherein the foldable central shape memory alloy component is securely received within an outer portion.

17. The gastric residence article as claimed in claim 16, wherein at least 80% of the folding force is attributed to the foldable central shape memory alloy component.

18. The gastric residence article as claimed in claim 16, wherein the linker component is coupled to the outer portion.

19. The gastric residence article as claimed in claim 16, wherein the outer portion is made of at least one selected from a group consisting of polypropylene, polystyrene, polyvinyl chloride, synthetic rubber, phenol formaldehyde resin (or Bakelite), neoprene, nylon, polyacrylonitrile, PVB, silicone, acrylonitrile butadiene styrene, high density polyethylene, polycarbonate, nylon, acrylic, polyethylene terephthalate, polybutylene terephthalate, acetal, polyimide, polyurethane, and epoxy.

20. A gastric residence article for oral administration comprising:
a foldable central component comprising a plurality of ribs, wherein the ribs comprise at least one superelastic alloy;
a plurality of elongated limb components radially attached to the foldable central component, the plurality of elongated limb components corresponding to the plurality of ribs; and
a plurality of linker components, each of the linker components connecting one of the elongated limb components to the foldable central component,
wherein the ribs are releasable from a folded first state to an unfolded second state for deploying the elongated limb components,
wherein at least one of the elongated limb components is configured to releasably store drugs, and
wherein the linker components are configured to transform from a rigid first state to a disengaged second state to allow the elongated limb component to move independently with respect to the foldable central component.

21. The gastric residence article as claimed in claim 20, wherein the disengaged second state comprises the linker components being in a flexible state to allow bending of the elongated limb components with respect to the foldable central component.

22. The gastric residence article as claimed in claim 20, wherein the disengaged second state comprises the linker components breaking apart to disconnect the elongated limb components from the foldable central component.

23. The gastric residence article as claimed in claim 20, wherein the linker components comprise a material that undergoes hydrolysis to transform the linker components from the rigid first state to the disengaged second state.

24. The gastric residence article as claimed in claim 20, wherein the superelastic alloy comprises at least one selected from a group consisting of Nitinol (Ni—Ti), Brass (Cu—Zn), copper-aluminium-nickel (Cu—Al—Ni) alloy, gold-cadmium (Au—Cd) alloy, gold-copper-zinc (Au—Cu—Zn) alloy, indium-thallium (In-TI) alloy, cobalt-nickel-aluminium (Co—Ni—Al) alloy, cobalt-nickel-gallium (Co—Ni—Ga) alloy, copper-aluminium-beryllium-zirconium (Cu—Al—Be—Zr), copper-aluminium-beryllium-chromium (Cu—Al—Be—Cr) alloy, copper-aluminium-beryllium-gadolinium (Cu—Al—Be—Gd) alloy, copper-aluminium-nickel-hafnium (Cu—Al—Ni—Hf) alloy, copper-tin (Cu—Sn) alloy, copper-zinc-silicon (Cu—Zn—Si) alloy, copper-zinc-aluminium (Cu—Zn—Al) alloy, copper-zinc-silicon (Cu—Zn—Sn) alloy, iron-manganese-silicon (Fe—Mn—Si) alloy, iron-platinum (Fe—Pt) alloy, manganese-copper (Mn—Cu) alloy, nickel-iron-gallium (Ni—Fe—Ga) alloy, (Ni—Ti—Hf) alloy, nickel-titanium-palladium (Ni—Ti—Pd) alloy, nickel-manganese-gallium (Ni—Mn—Ga) alloy, nickel-manganese-gallium-copper (Ni—Mn—Ga—Cu) alloy, nickel-manganese-gallium-cobalt (Ni—Mn—Ga—Co) alloy, titanium-niobium (Ti—Nb) alloy.

25. The gastric residence article as claimed in claim 20, wherein the superelastic alloy is a shape memory alloy.

26. The gastric residence article as claimed in claim 25, wherein the shape memory alloy comprises at least one selected from a group consisting of Ni—Ti, Ni—Ti—Hf, Ni—Ti—Pd, Ni—Fe—Ga, Ni—Mn—Ga, Ni—Mn—Ga—Cu, Ni—Mn—Ga—Co, Ag—Cd, Co—Ni—Al, Co—Ni—Ga, Cu—Al—Be—X (X: Zr, B, Cr, Gd), Cu—Al—Ni, Cu—Al—Ni—Hf, Cu—Sn, Cu—Zn, Cu—Zn—X (X=Si, Al, Sn), Fe—Mn—Si, Fe—Pt, Mn—Cu, Ti—Nb.

27. The gastric residence article as claimed in claim 20, wherein the foldable central component further comprises an outer portion and wherein the ribs are securely received within the outer portion.

28. The gastric residence article as claimed in claim 27, wherein the linker components connect each of the elongated limb components to the outer portion.

29. The gastric residence article as claimed in claim 27, wherein the outer portion is made of at least one selected from a group consisting of polypropylene, polystyrene, polyvinyl chloride, synthetic rubber, phenol formaldehyde resin (or Bakelite), neoprene, nylon, polyacrylonitrile, PVB, silicone, acrylonitrile butadiene styrene, high density polyethylene, polycarbonate, nylon, acrylic, polyethylene terephthalate, polybutylene terephthalate, acetal, polyimide, polyurethane, and epoxy.

30. The gastric residence article as claimed in claim 20 further comprising a casing for housing the foldable central component and the elongated limb components, wherein the casing is configured to be dissolved to release the ribs from the first folded state to the unfolded second state for deploying the elongated limb components.
